(19) United States
(12) Patent Application Publication
(10) Pub. No.: US 2023/0380740 A1
(43) Pub. Date: Nov. 30, 2023

(54) SENSOR-BASED TRAINING INTERVENTION

(71) Applicants: Agency for Science, Technology and Research, Singapore (SG); NATIONAL UNIVERSITY OF SINGAPORE, Singapore (SG)

(72) Inventors: Zheng Yang CHIN, Singapore (SG); Haihong ZHANG, Singapore (SG); Cuntai GUAN, Singapore (SG); Chuan Chu WANG, Singapore (SG); Tih Shih LEE, Singapore (SG)

(21) Appl. No.: 18/030,717

(22) PCT Filed: Oct. 7, 2020
(86) PCT No.: PCT/SG2020/050565

§ 371 Date: Apr. 6, 2023

(51) Int. Cl. A61B 5/16; A61B 5/378

(52) U.S. Cl. CPC A61B 5/163; A61B 5/378

(57) ABSTRACT

Disclosed is a system for sensor-based training intervention. The system includes one or more electroencephalogram (EEG) sensors for retrieving brain signals of a subject, one or more sensors for retrieving eye tracking data of one or both eyes of the subject, and one or more processors. The one or more processors are configured to model a joint state space of the brain signals and eye tacking data by combining the brain signals and eye tracking data into combined data using sequential Bayesian fusion, and measure a visuospatial attention indicator from combined data.

### TECHNICAL FIELD

The present invention relates, in general terms, to a system for sensor-based training intervention, and the method implemented on such a system. In particular, embodiments of the present invention relates to sensor-based training intervention for developing particular social behaviours.

### BACKGROUND

Autism Spectrum Disorder (ASD) is a pervasive neuropsychiatric disorder and the top cause of disease burden in children aged 14 and below, in Singapore and worldwide. This lifelong disorder, marked by deficits in social communication, interaction, and imagination, has an average prevalence of 1%. Children with ASD also present with severe functioning problems in day-to-day activities and are at an increased risk of developing depression, conduct disorders, and anxiety disorders.

There is no known cure nor generally approved medication for ASD. Current treatments involve primarily behavioural interventions with limited efficacy as they involve considerable effort and expense for the child and family. Evidence also suggests that early intervention may lead to better outcomes. However, many ASD children are diagnosed late.

Given these limitations, there is a need to explore alternative and novel approaches for early diagnosis and intervention which can lead to improvement in functioning even if a cure is not available.

It would be desirable to overcome or [alleviate/ameliorate] at least one of the above-described problems, or at least to provide a useful alternative.

### SUMMARY

Disclosed herein is a system for sensor-based training intervention including:
(a) one or more electroencephalogram (EEG) sensors for retrieving brain signals of a subject;
(b) one or more sensors for retrieving eye tracking data of one or both eyes of the subject;
(c) one or more processors configured to perform the following steps:
   i. modelling a joint state space of the brain signals and eye tacking data by combining the brain signals and eye tracking data into combined data using sequential Bayesian fusion; and
   ii. measuring a visuospatial attention indicator from combined data.

The visuospatial attention indicator is an indication or determination of the level of concentration of the subject on a point or points of interest.

The system may be employed to train social behaviour of the subject, a further comprise a display, wherein, in advance of steps (a) and (b), the display displays a social cue to the subject, and wherein step (c)ii. comprises measuring a visuospatial attention indicator associated with the social cue. Step (c)i. may comprise modelling a joint state space relating to the social cue.

The social behaviour may comprise interacting with the gaze of another person (the third party), and the display may then display the third party to the subject, and the social cue comprises one or both eyes of the third party. The eye or eyes of the third party may have a focus, and step (c)ii. may then comprise measuring a visuospatial attention indicator with reference to the focus. The one or more processors may be configured configured, at step (c)ii., to determine whether the subject is focussing on the focus of the third party. Determining whether the subject is focussing on the focus of the third party may comprise removing the social cue, wherein the one or more processors are configured to measure the visuospatial attention indicator based on whether the combined data infers recollection of the subject of the focus.

The social behaviour may comprise facial recollection, the display displaying a target face and, separately, a plurality of other faces, at least one said other face being the target face, and wherein the one or more processors are configured to measure the visuospatial attention indicator by determining if the subject focuses on the target face in the plurality of other faces.

The social behaviour may instead comprise facial expression recognition, the display displaying a scenario and the social cue, the social cue comprising a plurality of faces, each face of the plurality of faces expressing a response to the scenario, and wherein the one or more processors are configured to measure the visuospatial attention indicator by determining if the subject focuses on the face, of the plurality of faces, for which the response matches the scenario.

The system may be configured to be used repetitively, each subsequent repetition comprising displaying a more difficult or easier social cue depending on the visuospatial attention indicator of a previous repetition.

Also disclosed herein is a method for sensor-based training intervention, comprising:
(a) receiving brain signals from one or more electroencephalogram (EEG) sensors;
(b) receiving eye tracking data from one or more sensors;
(c) modelling, at one or more processors, a joint state space of the brain signals and eye tacking data by combining the brain signals and eye tracking data into combined data using sequential Bayesian fusion; and
(d) measuring a visuospatial attention indicator from combined data.

The method may be employed to train social behaviour of the subject, and further comprise displaying, in advance of steps (a) and (b), a social cue to the subject, wherein step (d) comprises measuring a visuospatial attention indicator associated with the social cue.

Step (c) may comprise modelling a joint state space relating to the social cue. The social behaviour may comprise interacting with the gaze of another person (the third party), and displaying the social cue may comprise displaying the third party to the subject, and the social cue comprises one or both eyes of the third party. The eye or eyes of the third party may have a focus, and the visuospatial attention indicator may be measured with reference to the focus. Step (d) may comprise determining whether the subject is focussing on the focus of the third party. Determining whether the subject is focussing on the focus of the third party may comprise removing the social cue, and measuring the visuospatial attention indicator based on whether the combined data infers recollection of the subject of the focus.

The social behaviour may instead comprise facial recollection, wherein displaying the social cue comprises displaying a target face and, separately, a plurality of other faces, at least one said other face being the target face, and measuring the visuospatial attention indicator by determining if the subject focuses on the target face in the plurality of other faces.

The social behaviour may instead comprise facial expression recognition, and the social cue is displayed in relation to a scenario, the social cue comprising a plurality of faces, each face of the plurality of faces expressing a response to the scenario, and measuring the visuospatial attention indicator comprises determining if the subject focuses on the face, of the plurality of faces, for which the response matches the scenario.

Steps (a) to (d) may be performed repetitively, wherein displaying the social cue for a repetition comprises displaying a more difficult or easier social cue depending on the visuospatial attention indicator of a previous repetition.

Advantageously, embodiment of the present invention combines eye tracker data and EEG data and models a combined feature space or state space from which it can be determined whether there is a point of interest on which the subject is focusing and/or whether the subject is focusing on a specific point of interest. From that determination, it can be inferred whether the subject understands a particular social cue with which they are presented.

More broadly, therefore, embodiments of the present invention enable the system or method to determine whether the subject is focusing on a point of interest that indicates they understand a social cue with which they are presented.

### BRIEF DESCRIPTION OF THE DRAWINGS

Embodiments of the present invention will now be described, by way of non-limiting example, with reference to the drawings in which:

FIG. 1 illustrates a method for sensor-based training intervention in accordance with present teachings;

FIG. 2 is a schematic diagram of a system for implementing the method of FIG. 1;

FIG. 3 is a further schematic diagram comprising the system of FIG. 2 and/or for implementing the method of FIG. 1;

FIG. 4 is a flow diagram showing steps in the performance of a method for sensor-based training intervention for training social behaviours;

FIG. 5 illustrates a way in which a user can receive feedback on how well they are focusing their attention on a target object;

FIG. 6 provide a sequence of displayed snapshots during performance of a gaze monitoring exercise for training the subject to interpret the gaze of a virtual avatar or human;

FIG. 7 shows a flow chart of computational steps performed during display of the sequence shown in FIG. 6;

FIG. 8 shows a display used for training the social behaviour of facial recognition;

FIG. 9 those an alternative display used for training the social behaviour of facial recognition, having an increased difficulty level over the display provided in FIG. 8;

FIG. 10 illustrates a progressive sequence of screenshots of another facial recognition exercise involving an element of memory retention of faces viewed in the immediate past;

FIG. 11 shows a display used for training the social behaviour of facial expression recognition;

FIGS. 12 to 18 illustrate various ways in which the difficulty level used for training a particular social behaviour may be adjusted depending on the performance of the subject; and

FIG. 19 is a system interaction diagram for implementing the FIG. 1 in the system such as that shown in FIG. 2 or 3.

### DETAILED DESCRIPTION

The systems and methods disclosed herein may measure the interactive focus of a subject (may also be referred to as a patient) on points of interest. Some prior art endeavours to achieve this by measuring electroencephalogram (EEG) signals and inferring concentration from those signals. However, the signals fail to take into account what the subject is looking at. For example, while concentrating, the gaze of a subject may move across multiple points of interest. Therefore, the subject will be concentrating but that concentration is placed on a thought rather than anything necessarily in the visual field of the subject. In other cases, the prior art focuses on tracking the eye movement of the subject and inferring, when the gaze lingers on a particular point, that the subject is focused on that particular point. However, the subject may not be concentrating at all.

In contrast, systems and methods disclosed herein seek to identify a joint feature space or joint state space of EEG and eye-tracker signals from which to infer a level of focus on points of interest. Thus, systems and methods disclosed herein may determine interactive focus on points within the subject's field of view.

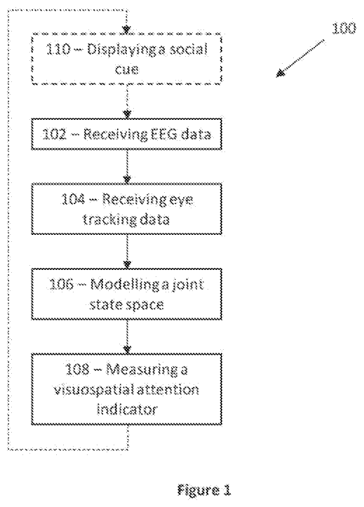

Such a method 100 is broadly defined in FIG. 1. That method 100 is for sensor-based training intervention, and comprises:
102: receiving brain signals from one or more electroencephalogram (EEG) sensors;
104: receiving eye tracking data from one or more sensors;
106: modelling, at one or more processors, a joint state space of the brain signals and eye tacking data by combining the brain signals and eye tracking data into combined data using sequential Bayesian fusion; and
108: measuring a visuospatial attention indicator from combined data.

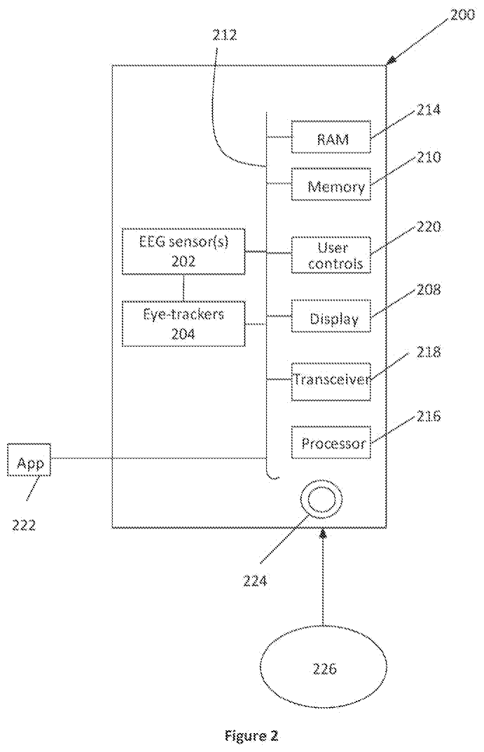

The method 100 may be employed, for example, on a computer system 200 as shown in FIG. 2. The block diagram of the computer system 200 will typically be a desktop computer or laptop. However, the computer system 200 may instead be a mobile computer device such as a smart phone, a personal data assistant (PDA), a palm-top computer, or multimedia Internet enabled cellular telephone.

As shown, the computer system 200 includes the following components in electronic communication via a bus 212:
(a) EEG sensors 202 for delivering the brain signals received at 102;
(b) eye trackers (sensors) 204 for delivering the eye tracking data received at 104;
(c) a display 208;
(b) non-volatile (non-transitory) memory 210;
(c) random access memory (“RAM”) 214;
(d) N processing components embodied in processor module 216, for performing steps 106 and 108;
(e) a transceiver component 218 that includes N transceivers; and
(f) user controls 220.

Although the components depicted in FIG. 2 represent physical components, FIG. 2 is not intended to be a hardware diagram. Thus, many of the components depicted in FIG. 2 may be realized by common constructs or distributed among additional physical components. Moreover, it is certainly contemplated that other existing and yet-to-be developed physical components and architectures may be utilized to implement the functional components described with reference to FIG. 2.

The main subsystems the operation of which is described herein in detail are the EEG sensors 202, the eye trackers 204, the one or more processors (i.e. N processing components) 216 and display 208. The sensors 202 and 204 measure a subject response to social cues or stimulate presented on display 208. The one or more processors 216 then interpret the data from the sensors 202 and 204 to measure a visuospatial attention indicator from which the correctness or otherwise of the subject response to the social cues can be inferred or determined. The display 208 may be realized by any of a variety of displays (e.g., CRT, LCD, HDMI, micro-projector and OLED displays).

In general, the non-volatile data storage 210 (also referred to as non-volatile memory) functions to store (e.g., persistently store) data and executable code, such as the instructions necessary for the computer system 200 to perform the method 100. The executable code in this instance thus comprises instructions enabling the system 200 to perform the methods disclosed herein, such as that described with reference to FIG. 1.

In some embodiments for example, the non-volatile memory 210 includes bootloader code, modem software, operating system code, file system code, and code to facilitate the implementation components, well known to those of ordinary skill in the art that, for simplicity, are not depicted nor described.

In many implementations, the non-volatile memory 210 is realized by flash memory (e.g., NAND or ONENAND memory), but it is certainly contemplated that other memory types may be utilized as well. Although it may be possible to execute the code from the non-volatile memory 210, the executable code in the non-volatile memory 210 is typically loaded into RAM 214 and executed by one or more of the N processing components 216.

The N processing components 216 in connection with RAM 214 generally operate to execute the instructions stored in non-volatile memory 210. As one of ordinarily skill in the art will appreciate, the N processing components 216 may include a video processor, modem processor, DSP, graphics processing unit, and other processing components. The N processing components 216 may form a central processing unit (CPU), which executes operations in series. In some embodiments, it may be desirable to use a graphics processing unit (GPU) to increase the speed of analysis and thereby enable, for example, the real-time assessment of visuospatial attention—e.g. during performance of a task. Whereas a CPU would need to perform the actions using serial processing, a GPU can provide multiple processing threads to perform processes in parallel.

The transceiver component 218 includes N transceiver chains, which may be used for communicating with external devices via wireless networks, microphones, servers, memory devices and others. Each of the N transceiver chains may represent a transceiver associated with a particular communication scheme. For example, each transceiver may correspond to protocols that are specific to local area networks, cellular networks (e.g., a CDMA network, a GPRS network, a UMTS networks), and other types of communication networks. In some embodiments, one or both of sensors 202 and 204 may be remote, rather than form components of the system as shown with reference to FIG. 30. The sensors 202 and 204 may then send data to the system via the transceiver component 218. In other embodiments, data from sensors 202 and 204 may be stored remotely and sent via the transceiver component 218 to the system, or the memory 210 may store that data.

Reference numeral 224 indicates that the computer system 200 may include physical buttons, as well as virtual buttons such as those that would be displayed on display 208. Moreover, the computer system 200 may communicate with other computer systems or data sources over network 226.

It should be recognized that FIG. 2 is merely exemplary and that the functions described herein may be implemented in hardware, software, firmware, or any combination thereof. If implemented in software, the functions may be stored on, or transmitted as, one or more instructions or code encoded on a non-transitory computer-readable medium 210. Non-transitory computer-readable medium 210 includes both computer storage medium and communication medium including any medium that facilitates transfer of a computer program from one place to another. A storage medium may be any available medium that can be accessed by a computer, such as a USB drive, solid state hard drive or hard disk.

To provide versatility, it may be desirable to implement the method 100 in the form of an app, or use an app to interface with a server on which the method 100 is executed. These functions and any other desired functions may be achieved using apps 222, which can be installed on a mobile device.

The system 200 may be more realistically presented in the network or system 300 shown in FIG. 3. The system 300 includes an eye tracker 302 (eye trackers 204) mounted in a display 304 (display 208—which displays the visual output of rehabilitation software), and an EEG headband 306 (EEG sensors 202). The tracker 302 and headband 306 measure data from a subject 308, and send that data to a workstation 310 that may house the transceiver component 218 processors 216 and other components of system 200. The workstation 310 may be a hybrid brain computer interface (BCI) client the processes the eye gaze data using eye gaze coordinates 312 from eye tracker 302 mounted, in the present instance, to display 304, and EEG signal 314 from EEG headband 306. Workstation 310 then produces a revised output for display on display 304, for BCI-based attention detection and eye gaze detection.

The system 300 can be employed to train social behaviour of the subject. To that end, in advance of performing steps 102 and 104, the display 304 will display a social cue to the subject 308. The workstation 310 then measures the visuospatial attention indicator associated with the social cue. This can involve modelling a joint state space relating to the social cue. This ensures the workstation 310 identifies features, or common features, in the EEG data and eye tracker data that are important for the particular social cue in question. In some cases, the joint state space may be a similar or the same state space for all social behaviour training programs.

The method 100, when performed in a system such as computer 200 or 300, produces a computerised system that tracks and visuospatial attention to train visual, memory and social skills of, for example, autistic children. The method and system enable customised social skills training to be delivered through software intended to target particular deficiencies in visual and social functions. These deficiencies include deficiencies in the ability of the subject to identify facial expressions and emotions, maintain eye-contact and interact with other people, and perform facial recognition.

As discussed below, the steps of the method may be performed repetitively, by displaying social cues at a repetition, and displaying a more difficult or easier social cue at any particular repetition depending on the visuospatial attention indicator of a previous repetition. As such, progressive training programs can be implemented. These training programs can range from guided (more easy) to unguided (more difficult) scenarios. The training programs may also arrange from abstract to more realistic scenarios where the user is first exposed to cartoons followed by realistic faces or human faces.

With further reference to system overview FIG. 3, the system 300 captures the frontal forehead EEG signals via the EEG headband 306 and the eye gaze positions of the subject via the eye tracker 302. The system 300 directs the subject 308 to look at and focus their visuospatial attention on targets of interest in the form of a customised training program displayed on display 304.

In general, customised training program will comprise a series of exercises (repetitions of the steps of method 100) integrated with the physiological measurements—i.e. data as measured by sensors 302 and 306. These exercises include maintaining eye contact, recognising facial expressions or emotions, and training the focusing of attention. The eye tracker 302 allows an accurate mapping of the subject's eyes onto the targets or points interest—in some embodiments a point of interest may be a target face, being a face the subject is being trained to recognise. Relatedly, the EEG headband 306 provides an objective measurement of the subject's attention level while looking at these targets.

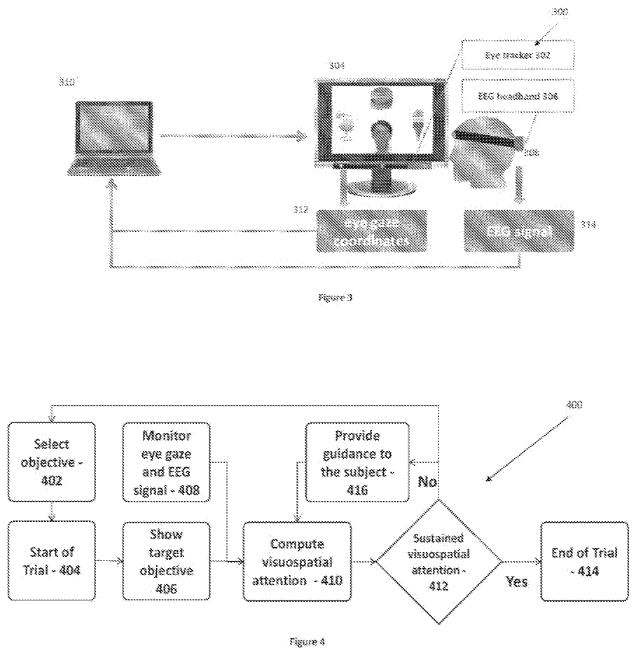

To improve engagement over delivery of standard starting material, the present system 200, 300 may gamify the delivery of method 100. The general gameplay mechanism is summarised in FIG. 4. This gameplay mechanism can be employed to a variety of social behavioural training scenarios.

Depending on the subject's progress, the subject will be presented with a target objective such as a virtual avatar's face on which to focus. At the start of each trial (repetition), the software embodying method 100 will display via display 208, 304 the target objective to the subject and continuously monitor the subject's visuospatial attention from the subject's eye gaze and brain computer interface (BCI) score—the BCI score will hereinafter be interchangeably referred to as the visuospatial attention indicator.

The general gameplay mechanism 400 involves the selection of appropriate objectives—e.g. social behavioural training objectives or programs, whether abstract or real—402. This may be done in an automated way, such as during a program in which a subject is tested on all available social behavioural programs in sequence, or in a manual way. After selection of appropriate objectives, the trial commences—404. Software implementing the method 100 shows the target objective to the subject—406. In some embodiments, the subject will be made aware of the nature of the exercise they are about to undertake—e.g. their ability to track the gaze of an avatar displayed to them—and in other embodiments they will be unaware of the exercise so that the system 200, 300 can determine whether the subject's response is a natural or learned one. During display of the target objective, the hybrid BCI (i.e. sensors 202, 204, 302, 306) monitors eye gaze positions of the subject and EEG signals of the subject—408. The processors 316 then measure the subject's visuospatial attention or indicator as computed from the combined EEG and eye tracker data—410.

Based on the visuospatial attention indicator, the system 200, 300 may determine whether the visuospatial attention of the subject was sustained on the target objective, for example a target face or object—412. It visuospatial attention was appropriately sustained, the trial ends—414. At this point, the difficulty of the objective may be increased, for example made more abstract, or the objective (the social behaviour being trained) may be changed. If visuospatial attention was not maintained or was below a desired threshold, guidance may be provided to the subject to help them focus—416. In some embodiments, the gamification mechanism may revert back to step 402 and select an easier objective for the subject to attempt, and perform a new trial.

An example of feedback delivered during performance of gameplay mechanism 400 is shown in FIG. 5. When the visuospatial attention indicator measured at step 412 is at or above a threshold, visual feedback is provided in the form of a circle that grows in size (from leftmost magnifying glass to rightmost magnifying glass), played in a magnifying glass. When the circle occupies the full magnifying glass, it means that the visuospatial attention indicator was at or above the predetermined threshold for a predefined time. As a consequence, a success action is registered—for example, the trial may end (414). If the subject's visuospatial attention indicator falls below the threshold before the circle occupies the full magnifying glass, the circle may either shrink in size or reset to 0 (leftmost magnifying glass). This is one of many potential ways of providing feedback to the subject in a real-time manner.

As mentioned above, one social behaviour that can be trained is the interaction between the subject and the gaze of another person (a third party or, presently, a virtual avatar). The social cue in this instance will be one or both eyes of the virtual avatar, which may include the direction the eyes are looking. The system 200, 300 may therefore provide a training scenario involving training the subject to focus on and/or follow the gaze of other people.

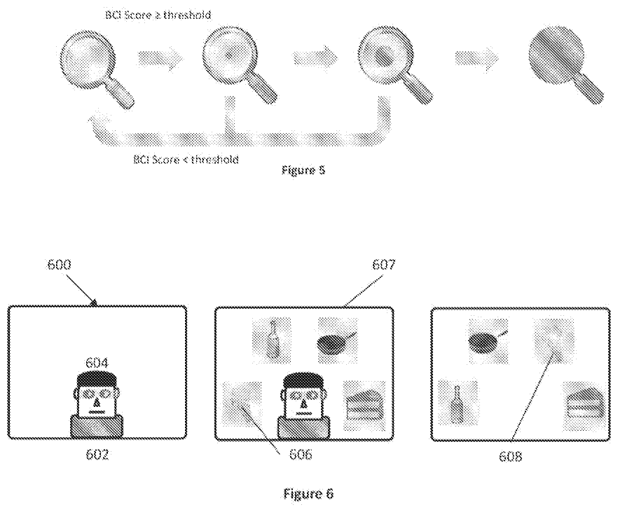

There are various mechanisms envisaged for achieving this. In one embodiment, the program trains the subject to process and follow eye gazes of another person, or other people. The program comprises one or more trials 600, each being divided into three parts as illustrated in FIG. 6. These three consecutive parts may be displayed concurrently though, for best results, should be displayed consecutively.

In the first part 602, the target objective is to identify the eyes of the virtual avatar 604. The subject is required to focus on the eyes. Presently, the eyes are looking in a particular direction as shown. The system 200, 300 may, using eye trackers, confirm whether or not the subject is focusing on a location of the display corresponding to the eyes of the virtual avatar 604.

In the second part 606, various objects are introduced into the field of view of the display—607. The direction of the eyes of the virtual avatar 604 remain unchanged. However, the eyes of the virtual avatar 604 now have a focus, namely the ice cream cone. The objective is to have the subject focus on the object at which the eyes of the virtual avatar 604 are looking. The system 200, 300 may therefore measure a visuospatial attention indicator with reference to the focus. For example, the system 200, 300 may determine whether the subject is looking at the object at which the virtual avatar is looking—i.e. whether the subject is focusing on the same thing as the virtual avatar.

In the third part 608, the virtual avatar is removed. The objective is to have the subject identify the same object, presently the ice cream cone, from a variety of objects displayed to the subject. Therefore, determining whether the subject is focusing on the same focus as that which the virtual avatar focussed on in 606, may involve removing the social cue (i.e. the avatar or their directional gaze) and determining whether the visuospatial attention indicator infers recollection of the subject (i.e. the ice cream cone) of the virtual avatar is focus.

The third part 608 may involve shuffling the objects, or introducing or replacing some objects with new objects. This increases the difficulty of recollection of the object on which the virtual avatar was focusing.

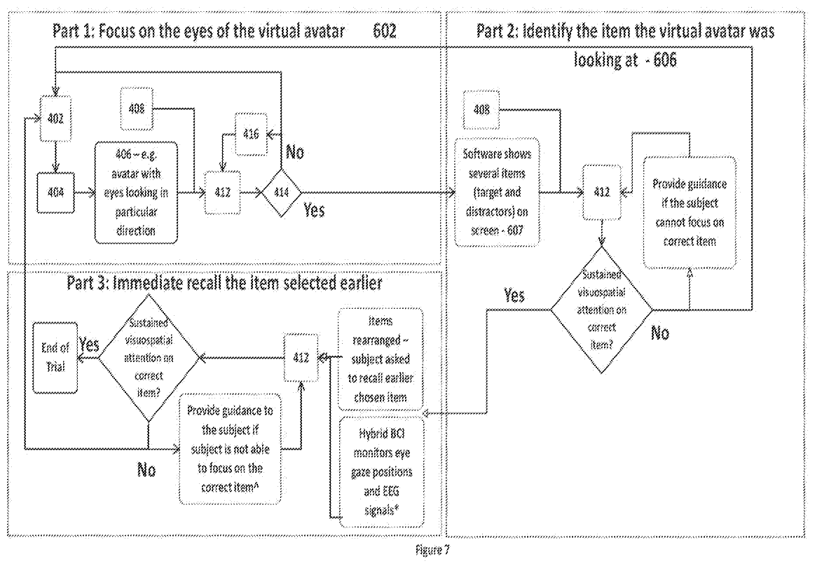

A flow chart 700 for illustrating the process of FIG. 6 is shown in FIG. 7. Part 1, referring to part 602 of FIG. 6, involves previously discussed steps 402 to 412 with the selected objective being that the subject focus on the virtual avatar 604 and, ideally, ascertain the direction of gaze of the eyes of the virtual avatar 604. Part 2, referring to part 606 of FIG. 6, repeats steps 406 to 412 with the software showing several objects including the target object, the ice cream cone in the example of FIG. 6, and distractors, namely objects towards which the virtual avatar is not gazing. Part three, corresponding to part 608 of FIG. 6, again repeats steps 406 to 412, with the virtual avatar removed and the software in some embodiments rearranging the objects to increase the difficulty of recollection of the target object by the subject. Upon successfully sustaining visuospatial attention on the correct object, trial ends per step 414 of FIG. 4. If the subject fails to sustain attention and any equivalent of step 412, the subject may be provided guidance feedback, to increase visuospatial attention on the requisite target, and/or the trial may restart either at the same difficulty, or at an easier difficulty.

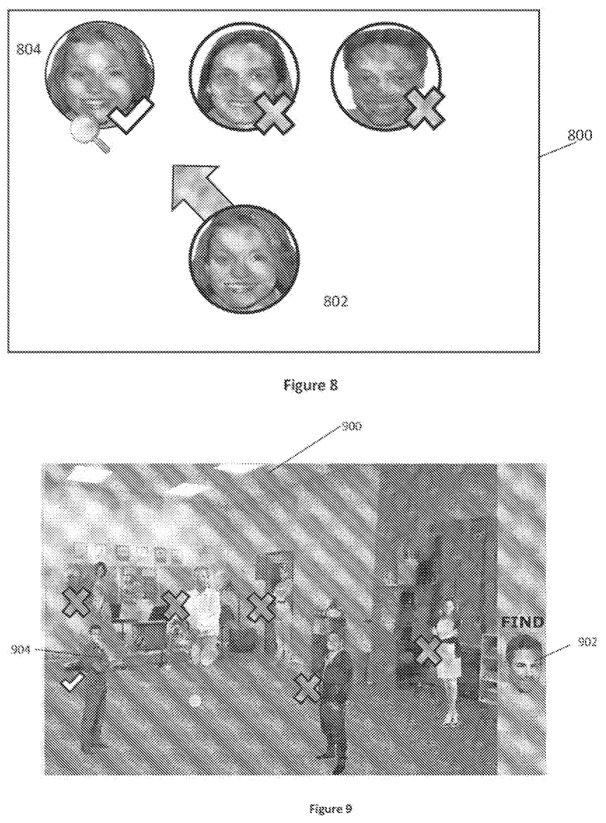

Another social behaviour that may be sought to be trained is facial recollection. Processing of faces is an important element for social interaction. However, individuals with Autism Spectrum Disorder often show a general face discrimination deficit. Accordingly, the system 200, 300 may include training programs (e.g. stored in memory 210) to train the subject to focus their attention and discriminate different faces as shown on the display—e.g. in a bubble launching game such as that shown in FIG. 8.

In this scenario, the subject focuses on one of a plurality of other, candidate, faces at the top of the display 800 that best matches a target face 802 at the bottom of the display 800. Presently, the best match is candidate face 804. Therefore, the system 200, 300 measures the visuospatial attention indicator by determining of the subject focuses on the target face in the plurality of other faces.

The scenario displayed in FIG. 8 is highly focused on ensuring the subject understands objective, by providing very little distracting information other than the candidate faces in the plurality of faces at the top of the display that do not match the target face 802. In the scenario displayed in FIG. 9, a display 900 displays a more realistic scene and a target face 902. The subject is expected to find the individual in the more realistic scene the face of whom most closely resembles the target face 902, presently individual 904.

Another training program for training facial recognition is shown in FIG. 10. The training program involves matching pairs of faces that are presented on a grid, and are then covered up. The subject is then required to flip cards or tiles of the grid over to find matching pairs of the previously displayed faces. To flip a card or tile, the subject must focus on the card or tile they desire to flip.

FIG. 10 shows progressive screenshots in implementation of the training program 1000. In particular, screenshot 1002 shows all faces of all tiles or cards sought to be matched. Screenshot 1004 shows the display immediately after all tiles or cards have been flipped over such that the faces can no longer be seen. Screenshot 1006 illustrates a stage in implementation of the program at which the subject has successfully identified two pairs of faces and has flipped over a card of a third pair sought to be matched. Finally, screenshot 1008 shows the display after all faces have been successfully matched.

Another social behaviour that may be sought to be trained is facial expression recognition. To train facial expression recognition, the display can be used to display a scenario and a social cue. For example, the scenario may be a picture or series of pictures or text designed to elicit an emotional response from a person. The social cue in this instance may be a plurality of faces each of which expresses a different response to the scenario, such as a different emotional expression—e.g. laughter, happiness, sadness or shock. The visuospatial attention indicator can then be measured by determining if the subject focuses on the face, of the plurality of faces, for which the response matches the scenario. An instance of such training as reflected in FIG. 11 that shows a display 1100, a plurality of faces 1102 each of which shows a different facial expression, and a box 1104 in which a particular social scenario is pictorially or textually set out. The subject is asked to review the scenario set out in box 1104 and to infer which facial expression, of those shown on faces 1102, best matches what a person or virtual avatar should be feeling given the description or particular social scenario. To determine which facial expression has been selected, the subject is required to focus on the face, of faces 1102, that shows the appropriate facial expression for the scenario.

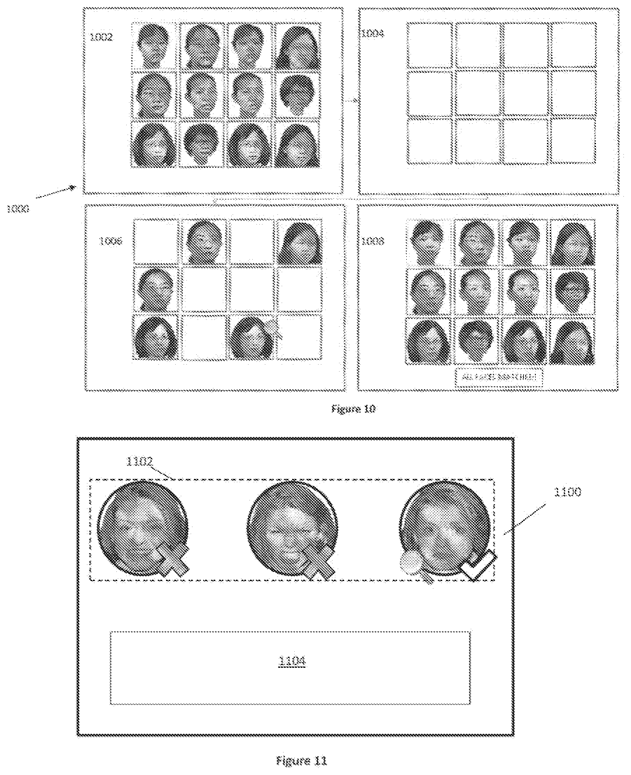

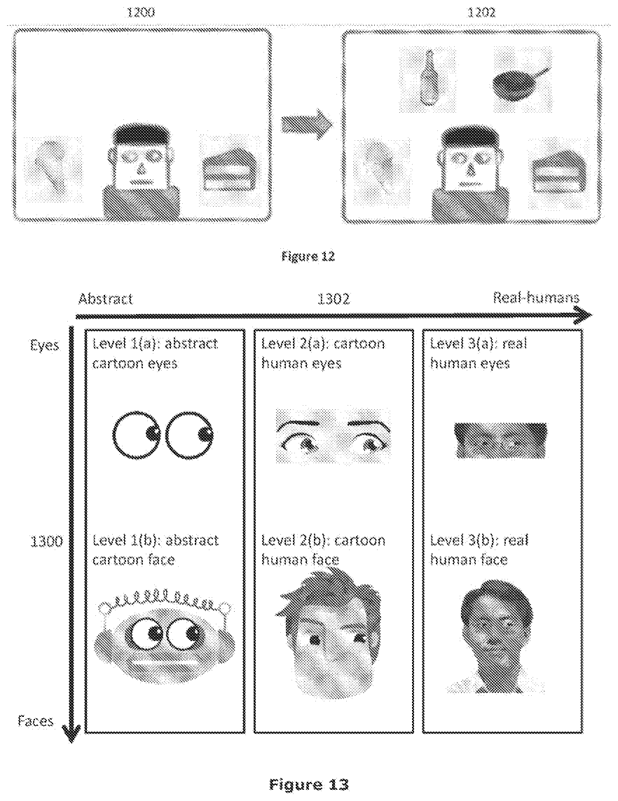

As mentioned above, the method 100, and consequently the system 200, 300 implementing that method, may provide progressive levels of difficulty to challenge and engage the subject based on their performance. To challenge the subject across multiple sessions during which they engage with the system 200, 300, the training exercises or trials employ a progressive level advancement structure. For example, when training the ability of the subject to follow the gaze of a virtual avatar, the number of objects in the visual field of the display may increase in more advanced levels, when the subject as shown aptitude in the social behaviour by correctly answering the earlier levels. In the example shown in FIG. 12, a first, easier implementation of the method is shown in display 1200 in which the subject need only between two objects, one of which the virtual avatar is gazing at. In a second, more difficult implementation of the method, shown in display 1202, the subject needs to select between four objects, with the avatar again only focusing on one of those objects.

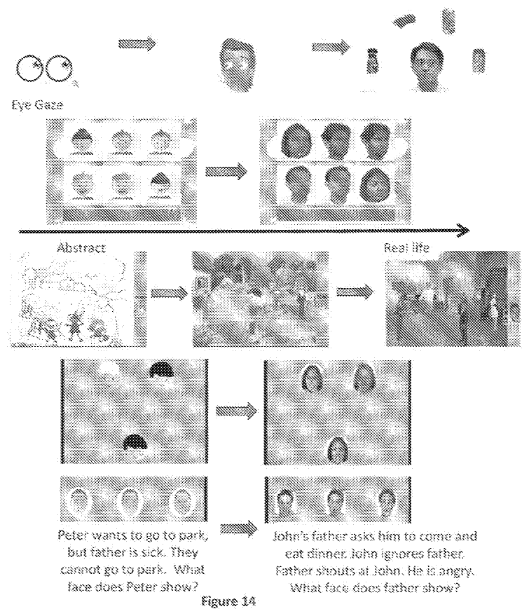

The method 100 may also progress from guided training to non-guided training. In earlier, easier levels, the exercises are designed to guide the subject step-by-step. An example of the progressive nature of the method 100 is shown in FIG. 13 to train the subject to follow the gaze of a displayed entity (e.g. cartoon, virtual avatar or human).

In more guided examples, only the eyes are shown and the other parts of the face are masked out to reduce the amount of distracting information presented to the subject. In the non-guided examples, the full face is shown and no further clues are given on where to focus. This is shown by the arrow 1300 indicating progressively increased difficulty as progressively more distracting information is introduced to the subject.

Progressively increased difficulty can also apply when transitioning from recognising a gaze of a set of cartoon eyes when compared with recognising the gaze of virtual human eyes. In earlier levels, abstract cartoon characters are used to attract the subject's attention. In later levels, pictures of real humans are used. The objective is to guide the subject towards familiarising themselves with real people in real life situations, and the manner in which people behave or facial information should be interpreted in real life situations. Increased difficulty in moving from more abstract representations to more real-life representations is indicated by arrow 1302.

FIG. 14 shows further scenarios in which various social behaviours are trained using programs having progressive difficulty increasing from left to right.

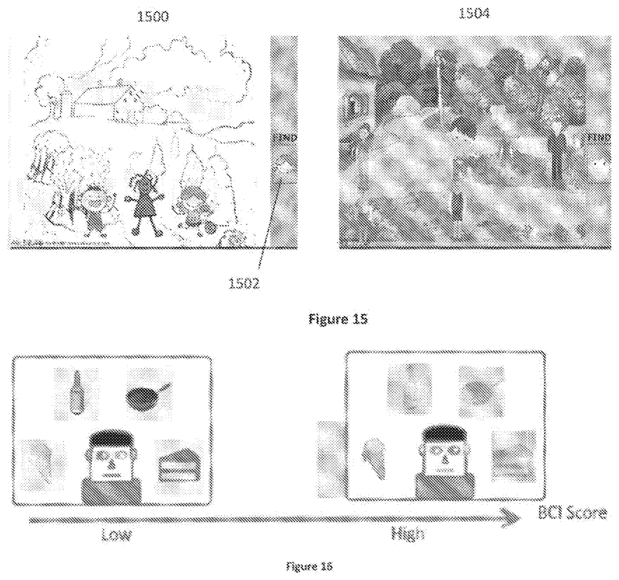

FIG. 15 shows another example of increasing difficulty in a facial recognition social behaviour training program. In the left-hand figure, display 1500, the subject is asked to find a target face 1502 in a black and white environment where candidate characters (i.e. those whose face may match the target face 1502) are shown in colour to attract the subject's attention. In the earlier levels, the subject is guided to focus only on the coloured characters to find a face matching the target face. In later levels such as that shown on display 1504, the environment becomes coloured, thereby increasing the amount of distracting information presented to the subject and reducing the distinct colour differences between the characters and the environment. As a consequence, the difficulty increases.

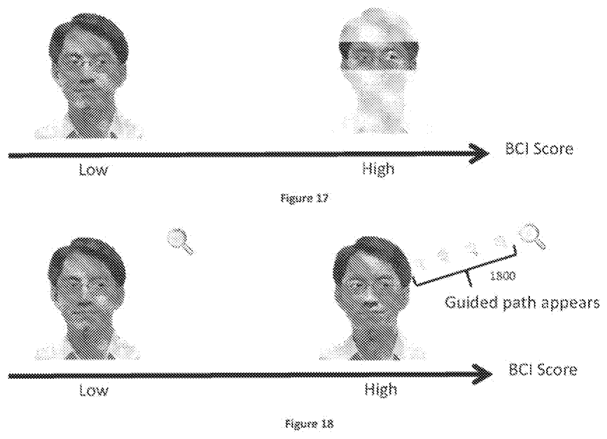

If the BCI score is high during gameplay, indicating a high degree of error, and the player continues to make mistakes in their selection of the appropriate object, facial expression or face match, the method 100 may guide the player towards a correct selection. As a consequence, the level of difficulty the creases with increased guidance. This can be done in several ways. Examples are shown in FIGS. 16 to 18 for the gaze track social behaviour. In FIG. 16, the non-target items or objects are masked out or faded when compared with the target item or object. In FIG. 17 parts of the face that comprise distracting information and do not assist the subject in determining the correct answer are masked out. In FIG. 18 a guided path 1800 appears showing the trajectory of the gaze of the cartoon, virtual avatar, human or other displayed entity. Thus for each of FIGS. 16 to 18, as the BCI score and thus the error made by the subject increases, increasing amounts of guidance are provided. It will be understood in view of present teachings that there are many ways in which the method 100 can modify the difficulty level of the program either in real-time or between successive trials or sessions with the subject, to progress the subject towards competence in the relevant social behaviour.

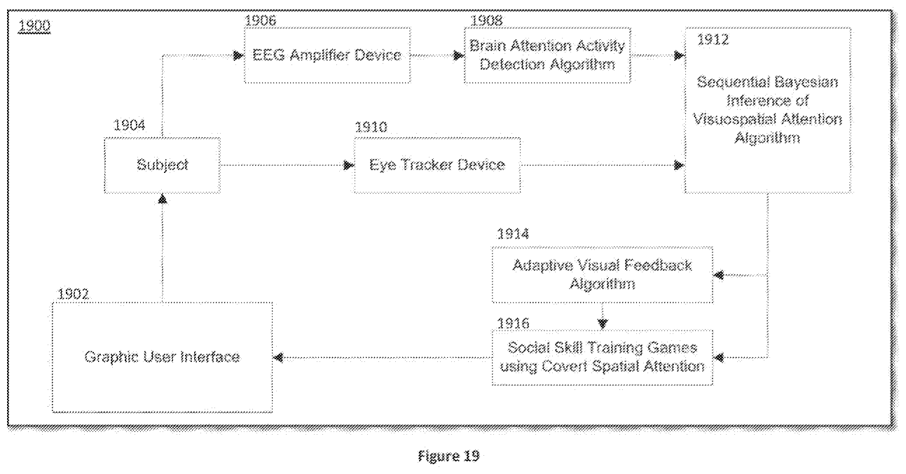

To combine the EEG data and eye tracker data, sequential Bayesian inference is proposed. FIG. 19 illustrates where that sequential Bayesian inference fits into the process flow 1900 of method 100 and systems 200, 300. A graphic user interface 1902 presents information to subject 1904. Eye tracking data is taken by device or sensors 1910. Concurrently, EEG of the subject are measured and amplified in device 1906 and passed through a brain attention activity detection algorithm at 1908. The output of brain attention activity detection algorithm 1908 and eye tracker device 1910 are fed into a module 1912 for sequential Bayesian inference of a visuospatial attention algorithm. The output of module 1912 is fed into an adaptive visual feedback algorithm 1914 that may provide feedback such as that shown in FIG. 5, and/or select or modify social skills training games using convert special attention at module 1916.

To perform sequential Bayesian infusion of EEG and eye tracker measurements, the following state space model for the visual spatial attention process is considered:

sₜ=Asₜ₋₁+nₛ (1)

where sₜ is a vector describing the visual attention state at time t, and sₜ₋₁ is the state at time t−1. The particular state vector presently proposed is described by Equation 2. A is the state transfer matrix that describes a linear transformation model of the visual attention state from time t−1 to t. nₛ is the stationary state process noise associated with the linear transformation model. In particular, the following visuospatial attention state vector is used:

s=[x,v,β] (2)

where x=[x, y, z] is the Cartesian coordinate triplet that defines the gaze position, v=[vₓ, v_{y}, v_{z}] is the linear speed of the gaze motion in the Cartesian space, and β is the score of attention.

This state space model is thus parametrised by A and nₓ only. The two parameters can be customised (e.g. through machine learning) to fit into different visuospatial attention processes. Described below is a basic example of the model parameters that fits visual attention processes as Gaussian processes, in which attention score β follows a random walk process and the motion of the gaze point following a smooth trajectory. Specifically, Matrix A takes the following form:

$$A = [ \begin{matrix}1 & 0 & 0 & Δ t & 0 & 0 & 0 \\ 0 & 1 & 0 & 0 & Δ t & 0 & 0 \\ 0 & 0 & 1 & 0 & 0 & Δ t & 0 \\ 0 & 0 & 0 & 1 & 0 & 0 & 0 \\ 0 & 0 & 0 & 0 & 1 & 0 & 0 \\ 0 & 0 & 0 & 0 & 0 & 1 & 0 \\ 0 & 0 & 0 & 0 & 0 & 0 & 1\end{matrix} ]$$

where Δt is simply the time interval between t and (t−1).

Now the state vector Sₜ is recursively estimated from a sequence of measurement data points in EEG and eye-tracker. We take a linear generative model that associates the state vector with the measurement vector uₜ:

uₜ=Bsₜ+nᵤ (4)

Here, uₜ contains measured variables from an EEG and/or eye-tracker, B is the mapping matrix that describes the transfer from state-space to measurement-space, and nᵤ is another stationary state process noise for the measurement noise. The measurement vector used for present purposes comprises two parts:

u=[\hat{x},w] (5)

The first part, \hat{x}, is the measured location of the gaze by the eye-tracker or sensor, and the second part, w, is the EEG representation vector of attentional state. Generally, w can be a combination of both temporal and spectral EEG features. Any validated EEG features can be used to represent the brain signals for attention. Presently, the feature extraction algorithm used in the presently described attention detection and training system generates the measurements or features.

A Guassian random variable model is then used for the noise component that is characterised by:

$${f}_{n} ( x ) = \frac{1}{\sqrt{{( 2 π )}^{k} \vert Σ \vert}} exp ( - \frac{1}{2} {( x - μ )}^{T} {Σ}^{-} 1 ( x - μ ) ) \qquad ( 6 )$$

where T is the transpose operator, μ the mean value, and Σ the covariance matrix.

The sequential Bayesian fusion algorithm is implemented in a series of steps. Firstly, the algorithm is initialised. That involves setting initial values for the state vector x₀ at time step t=0. Thereafter the algorithm moves to the next time point which, without loss of generality, is reflected by t+1->t. Subsequently, the measurement vector is computed from EEG and eye tracker data: uₜ. The initial prediction is then computed for the state vector using the state transition model under:

ŝₜ⁻=Aŝₜ₋₁; (7)

The a posteriori estimate of the error covariance is then made according to:

Pₜ⁻=APₜ₋₁A^{T}+Σₛ; (8)

The innovation matrix K is subsequently updated, where:

Kₜ=Pₜ⁻B^{T}(BPₜ⁻B^{T}+Σᵤ)⁻¹ (9)

The state vector estimate is then updated, where:

sₜ=sₜ⁻+Kₜ(uₖ−Bsₜ⁻) (11)

And the error covariance estimate is updated, where:

Pₜ=(I−KₜB)Pₜ⁻ (12)

The algorithm then repeats by incrementing the time step. Using this algorithm, the EEG data and eye tracker data may be fused (i.e. combined) to yield visuospatial attention representation of that data, they can be used to measure visuospatial attention indicator for the subject at the time the data was measured.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A system for sensor-based training intervention including:
(a) one or more electroencephalogram (EEG) sensors for retrieving brain signals of a subject;
(b) one or more sensors for retrieving eye tracking data of one or both eyes of the subject;
(c) one or more processors configured to perform the following steps:
i. modelling a joint state space of the brain signals and eye tacking data by combining the brain signals and eye tracking data into combined data using sequential Bayesian fusion; and
ii. measuring a visuospatial attention indicator from combined data.

2. The system of claim 1, being employed to train social behaviour of the subject, further comprising a display and wherein, in advance of steps (a) and (b), the display displays a social cue to the subject, and wherein step (c)ii. comprises measuring a visuospatial attention indicator associated with the social cue.

3. The system of claim 2, wherein step (c)i. comprises modelling a joint state space relating to the social cue.

4. The system of claim 2, wherein the social behaviour comprises interacting with the gaze of another person (the third party), and the display displays the third party to the subject, and the social cue comprises one or both eyes of the third party.

5. The system of claim 4, wherein the eye or eyes of the third party have a focus, and wherein step (c)ii. comprises measuring a visuospatial attention indicator with reference to the focus.

6. The system of claim 5, wherein the one or more processors are configured, at step (c)ii., to determine whether the subject is focussing on the focus of the third party.

7. The system of claim 6, wherein determining whether the subject is focussing on the focus of the third party comprises removing the social cue, wherein the one or more processors are configured to measure the visuospatial attention indicator based on whether the combined data infers recollection of the subject of the focus.

8. The system of claim 2, wherein the social behaviour comprises facial recollection, and the display displays a target face and, separately, a plurality of other faces, at least one said other face being the target face, wherein the one or more processors are configured to measure the visuospatial attention indicator by determining if the subject focuses on the target face in the plurality of other faces.

9. The system of claim 2, wherein the social behaviour comprises facial expression recognition, and the display displays a scenario and the social cue, the social cue comprising a plurality of faces, each face of the plurality of faces expressing a response to the scenario, and wherein the one or more processors are configured to measure the visuospatial attention indicator by determining if the subject focuses on the face, of the plurality of faces, for which the response matches the scenario.

10. The system of claim 2, being configured to be used repetitively, each subsequent repetition comprising displaying a more difficult or easier social cue depending on the visuospatial attention indicator of a previous repetition.

11. A method for sensor-based training intervention, comprising:
(a) receiving brain signals from one or more electroencephalogram (EEG) sensors;
(b) receiving eye tracking data from one or more sensors;
(c) modelling, at one or more processors, a joint state space of the brain signals and eye tacking data by combining the brain signals and eye tracking data into combined data using sequential Bayesian fusion; and
(d) measuring a visuospatial attention indicator from combined data.

12. The method of claim 11, being employed to train social behaviour of the subject, further comprising displaying, in advance of steps (a) and (b), a social cue to the subject, and wherein step (d) comprises measuring a visuospatial attention indicator associated with the social cue.

13. The method of claim 12, wherein step (c) comprises modelling a joint state space relating to the social cue.

14. The method of claim 12, wherein the social behaviour comprises interacting with the gaze of another person (the third party), and displaying the social cue comprises displaying the third party to the subject, and the social cue comprises one or both eyes of the third party.

15. The method of claim 14, wherein the eye or eyes of the third party have a focus, and wherein the visuospatial attention indicator is measured with reference to the focus.

16. The method of claim 15, wherein step (d) comprises determining whether the subject is focussing on the focus of the third party.

17. The method of claim 16, wherein determining whether the subject is focussing on the focus of the third party comprises removing the social cue, and measuring the visuospatial attention indicator based on whether the combined data infers recollection of the subject of the focus.

18. The method of claim 12, wherein the social behaviour comprises facial recollection, wherein displaying the social cue comprises displaying a target face and, separately, a plurality of other faces, at least one said other face being the target face, and measuring the visuospatial attention indicator by determining if the subject focuses on the target face in the plurality of other faces.

19. The method of claim 12, wherein the social behaviour comprises facial expression recognition, and the social cue is displayed in relation to a scenario, the social cue comprising a plurality of faces, each face of the plurality of faces expressing a response to the scenario, and measuring the visuospatial attention indicator comprises determining if the subject focuses on the face, of the plurality of faces, for which the response matches the scenario.

20. The method of claim 12, wherein steps (a) to (d) are performed repetitively, wherein displaying the social cue for a repetition comprises displaying a more difficult or easier social cue depending on the visuospatial attention indicator of a previous repetition.
